(19) United States
(12) Patent Application Publication
(10) Pub. No.: US 2021/0369497 A1
(43) Pub. Date: Dec. 2, 2021

(54) INSTANT COLD THERAPEUTIC APPARATUS

(71) Applicants: Hitesh SHAH, Mumbai (IN); Devarya H. SHAH, Mumbai (IN)

(72) Inventor: Devarya H. SHAH, Mumbai (IN)

(21) Appl. No.: 17/227,418

(22) Filed: Apr. 12, 2021

(30) Foreign Application Priority Data
May 29, 2020 (IN) 202021022574

(51) Int. Cl. A61F 7/02

(52) U.S. Cl. CPC A61F 7/02; A61F 2007/0087

(57) ABSTRACT

An instant cold therapeutic apparatus, comprising: at least one squeezable packet (1) associated with the apparatus, wherein the packet is gently squeezed by a user, a first burstable pouch (2) present inside the packet filled with first non-toxic reactant and a second burstable pouch (3) available inside the packet having a second non-toxic reactant, wherein the pouches present inside the packet include two non-toxic reactants, which react to produce cooling sensation at the infected portion of the user body.

### FIELD OF THE INVENTION

The present invention in general relates to the field of therapy/diagnosis. More particularly relates to an instant cold therapeutic apparatus, which is effective and quick in reducing numbing pain over a user's body. The apparatus is designed to chilled injuries by slowing down the flow rate of blood moving inside the user's body, which reduces swelling pain and further damages.

### BACKGROUND OF THE INVENTION

In medical terms, swelling is defined as enlargement of any organ, skin or other body portion which is generally caused by a buildup of fluids insides the tissues. The general case of swelling is caused due to bruises, insect bites, and repetitive strain injuries. The surplus fluid leads to a rapid increase in the swelling region over a small period of time (i.e. few hours to weeks). Swelling can occur all over a living body or only a specific region of the body such as hands, legs and lips.

Conventionally, for reducing the pain raised from swelling the cold things are applied to the specific portion of the body, such as an ice. In the market various kinds of cold pads are available and widely used by people for treating minor injuries. These cold pads are popular as well as considered as effective in treatments for easing the pain and swelling.

Some cold packs have gel materials that convert into an ice pack when stored in a freezer for few hours. According to a medical practitioner, cold packs should be used for treating sprained or twisted ankle issue or pulled muscle issue. Furthermore, the cold packs are also used over bruises, insect bites, and repetitive strain injuries, such as tendinitis.

Cold pads are known for cold therapy that helps an individual with muscle spasms, whiplash, and numerous procedures of arthritis as well. Cold therapy also eases inflammation and muscle spasms, two common sources of pain. Cold compression therapy also recognized as heliotherapy, that syndicates two of the principles of rest, ice, compression, elevation to decrease pain and puffiness from any aforementioned injury to soft tissues. Moreover, it is also recommended by orthopaedic surgeons for surgery.

Recurrent acquaintance to cold is accompanied by various kinds of health benefits. For instance, according to some publications, researchers have found evidence that exposure to cold speeds up metabolism. In an addition, the researchers also claim that athletes use ice packs and supplementary types of exposure to cold as a means to speed up recovery after physical exercise. Furthermore, cold body therapy is also interconnected to enhanced quality of sleep, more emphasized and even to a better-quality immune response.

In general terms, the ice pack or gel pack is a transferable soft bag filled with water, refrigerant gel, or liquid. For use the fillings are frozen in a freezer for few hours. Mutually ice and other non-toxic refrigerants (mostly water) is absorb a substantial quantity of heat formerly they warm above 0° C., due to the principle of high latent heat of fusion of water.

The working principle is water has an ample higher latent heat of fusion than utmost elements, and a melting temperature that is considered as convenient and effortlessly accomplished with, for example, a household freezer. Condiments to advance the properties of water are often used. For example, supplementary substances can be added to avert bacterial development in the pack, or to avert the water from coagulating so it remnants a thick gel during the course of use.

Gel packs are often used by people, which are ideally made of non-toxic materials that have a tendency to remain a slow-flowing gel, and consequently will not spill easily or cause adulteration if the container breaks. Gel packs are electively manufactured by adding hydroxyethyl cellulose (Cellusize), sodium polyacrylate, or vinyl-coated silica gel.

US20170122645A1 discloses about an improved freeze pack system that can instantly freeze any sports drink, electrolyte drink, or beverage into ice cold slush. The system comprises a pouch having central compartment and two compartments on the periphery of the pouch. The edible food item or beverage can be placed inside of the central compartment and the cold packs are placed inside the outer compartments that surround it from outside.

US20080141683A1 discloses about cold therapy is an established practice used in the medical profession to treat certain limb injuries, such as sprained or strained arm or leg muscles or injuries to joints. Generally, these types of injuries should be chilled to slow blood flow, which reduces swelling, pain, and further damage. Cold therapy is also an established practice used to treat migraine and other types of headaches. A typical course of cold therapy treatment is to apply ice for a specified period to the injured region of a limb, the neck, face, or head. Alternatively, a pack or bag containing a chemical agent that reacts (endothermically) to produce cold may be applied to the injured region, the neck, face, or head.

U.S. Pat. No. 5,792,213A discloses about hot or cold chemical pack (1) is provided that has two compartments (28, 30). One compartment (28) has a natural weak point (32) or seam (at 24 or 26) and is contained inside the other compartment (30). Each compartment contains a chemical reactant. In one embodiment, the outer compartment (30), which is made of a flexible, vinyl or plastic material, is arranged in a semi-collapsed configuration. Each end of the inner compartment (28) is fixedly attached to a respective end (24, 26) of the outer compartment (30). The pack (10) is chemically activated by pressing on, kneading, or twisting the outer compartment (30), or pulling on each end of the pack (10). The lateral, outward pressure caused by the pressing on, kneading, or twisting of the pack, or by the user's pulling on the ends of the pack, is exerted at the rupturable seam (32) of the inner compartment (28). At a predetermined pressure, the natural weak point (32) or seam is ruptured and the reactant contents of the two compartments merge (34), which creates the heating (exothermic) or cooling (endothermic) chemical reaction. Consequently, the hot or cold therapeutic pack (10) is more easily activated than existing chemical therapy packs, which makes this pack (10) more convenient to use.

U.S. Pat. No. 4,081,256A discloses about particulate composition producing an endothermic reaction when mixed with water, and a cold pack containing such a composition, and water isolated therefrom until the intended time of use. The composition consists essentially of, in parts by weight, about 65 to about 130 parts urea, about 35 to about 80 parts hydrated sodium acetate (Na C₂ H₃ O₂.3H₂ O), about 18 to about 40 parts potassium chloride, potassium nitrate, or mixtures thereof, about 18 to about 30 parts ammonium chloride, and about 6 to about 10 parts guar gum. A cold pack comprises an outer sealed, flexible, impermeable enclosure containing the composition, and an inner sealed, flexible, impermeable, easily ruptured enclosure containing about 85 to about 140 parts by weight water. The pack reaches and maintains a temperature of about −7° C. for up to 30 minutes, and is moldable to conform to the surfaces being cooled.

CN103405305A discloses an instant ice bag for nationwide fitness programs and sport medicine, belonging to the technical field of ice bag design. The instant ice bag comprises an outer plastic bag, a coolant, an inner plastic bag and water liquid, wherein the water liquid is sealed into the inner plastic bag; the coolant and the inner plastic bag filled with the water liquid are sealed into the outer plastic bag; water liquid in the inner plastic bag is separated from the coolant through the inner plastic bag. The instant ice bag is characterized in that the coolant is a particle material prepared by stirring and mixing ammonium nitrate and urea; the inner plastic bag is a thinner bag body; the weight ratio of the coolant to the water liquid in the inner plastic bag is 1:1; the weight percentages of two components, namely the ammonium nitrate and urea in the coolant are respectively 75% and 25%; the water liquid is packed into the inner plastic bag in a hot melting and press fit mode; a fixed belt is also arranged, one end of the fixed belt is fixedly connected with the inner plastic bag, and the other end is fixedly connected with the outer plastic bag.

U.S. Pat. No. 3,893,834A discloses an improvements in refrigerating and heating packages (cold packs and hot packs) of the type containing a dry chemical in the outer main package and an additional separate quantity of water mixable to produce an endothermic or exothermic reaction respectively; multi-cell cold and hot packs with insulating means to retain the cold or heat of the activated cell(s) against non-work affecting waste or leakage; single cell hot and cold packs with insulating means to minimize temperature leakage and protect the user; integral wrap-around insulating means for cold and hot packs to hold same against the work being heat exchanged and minimize heat leakage; mitten-type insulation sleeves integral with single or multiple cell hot and cold packs for positioning of the packs with respect to the user's hands, wrists, forearms, feet, ankles and the like; multi-cell hot and cold packs for wrap-around heat exchange of a work object with cooperating heavy stock labels operative to encircle the activated packs, retaining same in connection with the work and performing an insulating function.

U.S. Pat. No. 4,041,203A non-woven fabric-like material comprising an integrated mat of generally discontinuous, thermoplastic polymeric microfibers and a web of substantially continuous and randomly deposited, molecularly oriented filaments of a thermoplastic polymer. The polymeric microfibers have an average fiber diameter of up to about 10 microns while the average diameter of filaments in the continuous filament web is in excess of about 12 microns. Attachment between the microfiber mat and continuous filament web is achieved at intermittent discrete regions in a manner so as to integrate the continuous filament web into an effective load bearing constituent of the material. The material has desirable strength characteristics and possesses a textile-like appearance, drape and hand, By autogenously bonding the mat and web together in a manner so as to provide substantially uniform discrete bond regions, particularly outstanding strength characteristics with respect to energy absorption, tensile strength, and tear resistance can be achieved.

Several instant cold or ice pack devices or apparatuses are developed and widely popular in the market which comprises mixture of at least two reactants for developing cold sensation. Yet, there is no apparatus that have the capability to generate cooling sensation at very fast rate. Moreover, it can be used for prolonged cooling.

In order to overcome the aforementioned drawbacks, there is a need to develop an instant cold therapeutic apparatus that have features like speedy reaction and better cooling results and made up of a non-toxic, sustainable and biodegradable material, reduces the plastic waste as well as have prolonged duration of cooling, and flimsy weight.

### OBJECTS OF THE INVENTION

The principal object of the present invention is to overcome the disadvantages of the prior art.

An object of the present invention is to provide an instant cold therapeutic apparatus that utilizes non-poisonous, sustainable and decomposable material.

Another object of the present invention is to provide an apparatus, which produces prolonged cooling effect by using urea as a starting reactant for carrying out a reaction.

Another object of the present invention is to provide an apparatus that enhances the rate of reaction for producing instant cooling sensation.

Yet another object of the present invention is to provide an apparatus, which is environment friendly in nature and minimizes the need of plastic material.

The foregoing and other objects, features, and advantages of the present invention will become readily apparent upon further review of the following detailed description of the preferred embodiment as illustrated in the accompanying drawings.

### SUMMARY OF THE INVENTION

The present invention relates to an instant cold therapeutic apparatus that have features like speedy reaction and better cooling results and made of non-toxic, sustainable and degradable material, and reduces the plastic waste as well as have prolonged duration of cooling.

According to an embodiment of the present invention, an instant cold therapeutic apparatus is directed to produce cooling sensation to slow down the flow rate of blood moving inside the user body so as to minimize the swelling pain and other damages. The apparatus, comprising: a packet act as outer covering of the apparatus which is directly applied on the affected portion of the body to produce cooling sensation on the same. The packet is made up of non-toxic, eco-friendly and biodegradable material which make it reusable in nature.

The apparatus further comprises: two pouches of similar rectangular symmetry. Both the pouches are present inside the packet, when an external force is applied on the packet by the hand of the user, the inner pouches burst to produce cooling sensation. The primary pouch includes non-toxic reactant which is widely used as a good fertilizer as a source of nitrogen.

The secondary pouch is present inside the packet, which includes inorganic solvent, such as water. The non-toxic solvent from primary pouch and inorganic solvent from secondary pouch react together to produce ammonia solvent and carbon-dioxide. Both the output product combines together to produce reactants. The reaction is endothermic in nature.

While the invention has been described and shown with particular reference to the preferred embodiment, it will be apparent that variations might be possible that would fall within the scope of the present invention.

### BRIEF DESCRIPTION OF THE DRAWINGS

These and other features, aspects, and advantages of the present invention will become better understood with regard to the following description, appended claims, and accompanying drawings where:

FIG. 1 illustrates a front view of an instant cold therapeutic apparatus in accordance with an embodiment of the present invention; and

FIG. 2 illustrates a front view of two pouches in accordance with an embodiment of the present invention.

### DETAILED DESCRIPTION OF THE INVENTION

The following description includes the preferred best mode of one embodiment of the present invention. It will be clear from this description of the invention that the invention is not limited to these illustrated embodiments but that the invention also includes a variety of modifications and embodiments thereto. Therefore, the present description should be seen as illustrative and not limiting. While the invention is susceptible to various modifications and alternative constructions, it should be understood, that there is no intention to limit the invention to the specific form disclosed, but, on the contrary, the invention is to cover all modifications, alternative constructions, and equivalents falling within the spirit and scope of the invention as defined in the claims.

The present invention relates to an instant cold therapeutic apparatus for treating bruises, insect bites, and repetitive strain injuries, such as tendinitis in quick time and is non-toxic, sustainable, biodegradable in nature to minimize the plastic waste as well as have protracted period of cooling.

The apparatus is fabricated to decline the flow rate of body fluid, such as blood during the injuries which is helpful in reducing the swelling pain and further damages. It is having rectangular configuration and made up of non-toxic and biodegradable material which makes it reusable in nature. As the apparatus includes two different compartments, such as first and second compartments. Both the compartments are having similar configuration. They are present inside the enclosure. The one of the compartments known as “first pouch” comprises non-toxic reactant and on the other hand the other compartment which is known as “second pouch” includes inorganic solvent as a second reactant. The two reactants combine together to produce cooling sensation on the affected area of the body.

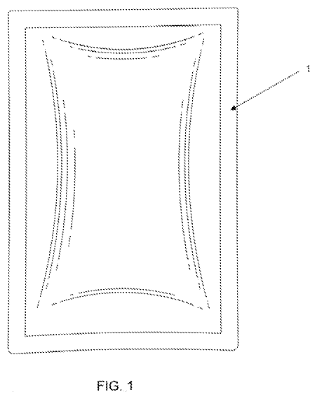

Referring to FIG. 1, a front view of an instant cold therapeutic apparatus is illustrated herein. The apparatus comprises a packet (1) (enclosure) and two pouches (2), (3) (inner compartments).

The apparatus is having rectangular shape and flimsy in nature, which make it easy to carry from one place to another. It is made up of a safe, non-toxic, and biodegradable material. It is only applied on the external body part of the user. It works on the amount of external force applied by the user hand which further helps in bursting of two pouches containing two reactants to give cooling sensation on the injured area.

The apparatus comprises packet having length of about 250 millimeter and breadth of about 166 millimeter. When the user put the packet on his/her hand then he or she can squeeze the packet with his or her palm by applying normal hand force of about 20-35 kgs/cm². Once the external force is applied the inner pouches open itself and activate the apparatus, so as to produce relief on the injured portion of the user body.

The packet (1) comprises two pouches (2), (3) of almost similar geometry. Both the pouches are arranged in an opposite manner to each other. Due to which when the user squeezes the packet with his/her hand force the two pouches present inside the packet bombard with each other. When both the pouches bombard with one another then the poches burst. The reactants present inside the both pouches come in contact to each other to produce the product which is responsible for producing the cooling sensation of the affected portion of the user body.

The first pouch (2) is present on the right side inside the packet is having length of about 90 millimeter and breadth of about 95 millimeter. The first packet comprises a first component. The first component is non-toxic and eco-friendly in nature. It is also known as “nitrogen containing compound” and used as a fertilizer as a source of nitrogen. It is also used as important raw material for chemical industry. The first component is preferably urea having a linear formula (NH₂—CO—NH₂). The urea consists of two amine groups and one carbonyl group.

The urea is colorless, odorless solid, highly soluble in water, and non-toxic in nature. It is neither acidic nor alkaline in nature. It is also known as “Carbamide”.

The second pouch (3) is present inside the left side of the packet adjacent to the urea containing pouch. It is having length of about 90 millimeter and breadth of about 95 millimeter. The pouch comprises inorganic solvent as a second reactant. The inorganic solvent is selected from water, sulfur dioxide, sulfuryl chloride, phosphoryl chloride, and dinitrogen tetroxide, preferably water.

The water includes two atoms of hydrogen and single atom of oxygen, covalently combined with each other. It exists in three states, such as solid, liquid and gases. It is also known as “Oxidane”. It has boiling point of about 100 degree Celsius and density of about 997 kg/m³.

The two reactants are present inside the two pouches which are available inside the packet combine with each other to form ammonia gas and carbon-dioxide gas. It is generally an endothermic reaction as the heat is absorbed during the reaction.

The reaction between two reactants are reversible in nature. As when both the urea and water react with each other to form carbon dioxide and water. The output products again combine with each other to produce the two reactants, such as urea and water. The reaction between both the reactants is represented below:

NH₂—CO—NH₂(aq)+H₂O(l)=NH₃(g)+CO₂(g) (Reversible in nature)

The two reactants are mixed in the ratio of 1:3. The prolonged cooling effect is produced by adding phosphate with reactants which is mainly responsible for prolong cooling effect. The fine granular size of urea as a reactant present inside the right pouch shows the speedy reaction and better cooling effect. In order to enhance the speed of the reaction the ammonium salt can be added during the reaction between two reactants, such as urea and water.

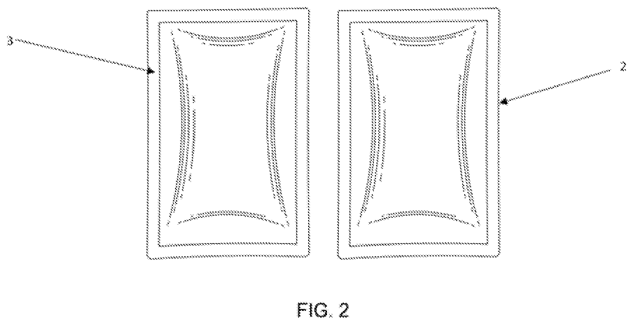

Referring to FIG. 2, a front view of two pouches are illustrated herein. The two pouches are available inside the packet. The two pouches are made up of material which burst on simply squeezed on applying hand force of about 20-35 kgs/cm².

The two pouches comprise two reactants which combine together to reduce the rete of flow of blood present inside the body of the user. Both the reactants include different elemental atoms covalently attached to each other.

The two pouches are having dimension lesser than that of the packet. The reaction between two reactants are responsible for producing instant cooling sensation on the applied portion of the user, which at the end helps the user to get relief from the pain. It can be applied on any of the external portion of the user body.

The apparatus is designed to slow down the flow rate of body fluid moving inside the user body. Once the reactants present in two different pouches come in contact with each other they break the previous bonds so as to form new bonds. When both the reactants breakdown they produce the cooling sensation which play a vital role in stopping the passage of the body fluid moving inside the body, at the end it provide relief to the user from the pain. In another embodiment of the present invention, the instant cold pack can be used for cooling beverages and food items.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically by interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances, where a convention analogous to “at least one of A, B, or C, etc.” is used, in general such a construction is intended in the sense one having skill in the art would 11 of 6 understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It will be further appreciated that functions or structures of a plurality of components or steps may be combined into a single component or step, or the functions or structures of one-step or component may be split among plural steps or components.

The present invention contemplates all of these combinations. Unless stated otherwise, dimensions and geometries of the various structures depicted herein are not intended to be restrictive of the invention, and other dimensions or geometries are possible. In addition, while a feature of the present invention may have been described in the context of only one of the illustrated embodiments, such feature may be combined with one or more other feature of other embodiments, for any given application.

It will also be appreciated from the above that the fabrication of the unique structures herein and the operation thereof also constitute methods in accordance with the present invention. The present invention also encompasses intermediate and end products resulting from the practice of the methods herein. The use of “comprising” or “including” also contemplates embodiments that “consist essentially of” or “consist of” the recited feature.

Although embodiments for the present subject matter have been described in language specific to structural features, it is to be understood that the present subject matter is not necessarily limited to the specific features described. Rather, the specific features and methods are disclosed as embodiments for the present subject matter. Numerous modifications and adaptations of the system/component of the present invention will be apparent to those skilled in the art, and thus it is intended by the appended claims to cover 12 of 6 all such modifications and adaptations which fall within the scope of the present subject matter.

I/We claim:

1. An instant cold therapeutic apparatus, comprising:
at least one squeezable packet (1) associated with said apparatus, wherein said packet is gently squeezed by a user;
a first burstable pouch (2) present inside said packet filled with non-toxic reactant as a first reactant, where said non-toxic reactant is a nitrogen containing compound; and
a second burstable pouch (3) residing inside said packet having inorganic solvent as a second reactant, wherein said pouches burst inside said packet while pressing for mixing out said reactants so as to provide instant cooling sensation to the applied region of said user's body.

2. The apparatus as claimed in claim 1, wherein said packet (1) have dimensions similar to a palm of said user who is responsible to smooth out said gentle squeezing of said packet.

3. The apparatus as claimed in claim 2, wherein said squeezing ideally ranges between 20-35 Kgs/cm2 of said user hand force.

4. The apparatus as claimed in claim 1, wherein said nitrogen containing compound is preferably urea.

5. The apparatus as claimed in claim 1, wherein said inorganic solvent is selected from group consisting of water, sulfur dioxide, sulfuryl chloride, phosphoryl chloride, and dinitrogen tetroxide.

6. The apparatus as claimed in claim 1, wherein said mixing ratio of said reactants during reaction is 1:3. Contain phosphate in a small along with urea.

7. The apparatus as claimed in claim 1, wherein said urea shows quick reaction in order to achieve better cooling results.
